(19) United States
(12) Patent Application Publication
(10) Pub. No.: US 2009/0156915 A1
(43) Pub. Date: Jun. 18, 2009

(54) Glucose Monitor and Method of Use Thereof

(76) Inventor: Bill Cross, Waco, GA (US)

Correspondence Address: WILLIAMSON INTELLECTUAL PROPERTY LAW, LLC, 1870 THE EXCHANGE, SUITE 100, ATLANTA, GA, 30339, US

(21) Appl. No.: 12/370,981

(22) Filed: Feb. 13, 2009

(51) Int. Cl. A61B 5/1455

(52) U.S. Cl. 600/316

(57) ABSTRACT

An apparatus for monitoring glucose comprising a processor, an indicating mechanism and sensors that are disposed proximate the skin of a person when the skin is in contact with a motor vehicle operational component such as the steering wheel. The apparatus for monitoring glucose measures the driver's glucose concentration via optical coherence tomography or other non-invasive technique, analyzes the driver's glucose concentration via the processor and displays the driver's glucose concentration via the indicator, or alternately sends an alarm signal. The method for monitoring glucose further comprises programming the processor with a range of glucose concentrations, comparing the driver's glucose concentration with the range and signaling an alert if the driver's glucose concentration is outside the range.

### CROSS-REFERENCE TO RELATED APPLICATIONS

None

### FEDERALLY SPONSORED RESEARCH OR DEVELOPMENT

None

### PARTIES TO A JOINT RESEARCH AGREEMENT

None

### REFERENCE TO A SEQUENCE LISTING

None

### BACKGROUND OF THE INVENTION

1. Technical Field of the Invention

The preferred embodiment relates generally to a glucose monitor and method of use thereof, and more specifically to a glucose monitor comprising a processor, an indicating mechanism and at least one sensor disposed proximate the skin of a person when the skin is in contact with a motor vehicle operational component.

2. Description of Related Art

Diabetes mellitus is a disorder of carbohydrate metabolism resulting from insufficient production of insulin and/or reduced sensitivity to insulin. In persons who have diabetes, the normal ability of body cells to utilize glucose is inhibited, thereby leading to abnormally high blood sugar levels, which may cause a variety of medical complications. Such complications include a condition known as diabetic retinopathy (retinal changes leading to blindness), kidney disease and frequent infection. Additionally, complications from diabetes may be fatal and include instances wherein a diabetic is driving a motor vehicle and goes into “diabetic shock,” thereby losing consciousness while driving, resulting in potentially fatal car accidents.

Aside from the possibility of a diabetic going into “diabetic shock,” persons with diabetes face a variety of issues while driving. Diabetics are unable to determine their current glucose levels or changes in their glucose levels while driving because it may be unsafe to simultaneously drive and check their glucose concentration. Similarly, if and when changes occur in a person's glucose level, the person does not know if they are capable of operating a vehicle safely. Lastly, if a diabetic has difficulty operating a vehicle and encounters a police officer, a diabetic may be unable to explain that he or she is suffering from either abnormally low or high glucose levels, and, in fact, the diabetic may not even know that they are so suffering. Accordingly, police officers are often unable to assist diabetics if medical attention is necessary, or mistakenly come to the conclusion that a diabetic is under the influence of drugs or alcohol.

One particular disadvantage is driving by commercial motor vehicle (CMV) licensed drivers who are under stringent controls both in the U.S. and other countries and who have a greater need than the average diabetic driver, since they need to apply for an exemption because of their diabetic condition. When a person holding a CMV license is diagnosed with diabetes they no longer are able keep their CMV license status without applying for the Federal Diabetes Exemption Program. The Exemption Program application process is very lengthy and involved and must be repeated every two years. Besides having to do a good deal of paperwork, the applicant has to undergo extensive medical exams every year before the exemption will even be considered. Further, the U.S. exemption does not apply for foreign countries, and it is often practice that a driver may have to continue his/her journey in Canada or Mexico.

There are a variety of treatments aimed at controlling diabetes, such as, placing patients on restrictive diets designed to help them reach and maintain normal body weight and to limit their intake of carbohydrates and fats. Another treatment available for diabetics is injections, wherein a diabetic receives regular injections of insulin. Further, medications have been employed to help maintain a diabetic's blood glucose levels within target ranges. However, while such treatments are helpful in controlling diabetes, such treatments fail to inform a diabetic as to their current glucose level and/or changes in their glucose levels over a period of time.

Currently, there are a variety of methods and devices available to determine a person's blood glucose level. One such method includes removing a sample of blood and performing chemical tests. Another method includes pricking a diabetic's finger (which is quite painful) in order to draw blood to place on a test strip, which is then inserted into an electronic glucose measuring device, resulting in the test strip changing color based on the level of glucose present in the blood. The color changes are then detected by the device and results are displayed on the measuring device. However, while such methods and devices are helpful in determining a person's blood glucose levels, such methods and devices may require trained technicians to remove blood from the diabetic and/or perform chemical tests, which may be time consuming. Further, many diabetics are reluctant to have either their finger pricked or have blood samples removed due to concern over the possibility of infection, discomfort and/or generalized patient fear.

Luckily, there are a variety of methods available to determine a person's blood glucose level in a non-invasive manner. Such procedures include nuclear magnetic resonance (NMR), electron spin resonance (ESR) and infrared spectroscopy, which are spectroscopic techniques utilized to infer the properties of bio-molecules via angular momentum. However, while such methods eliminate the need to extract blood, they require large and costly equipment and are unsuitable for routine analysis and/or patient self-checking.

Additionally, a variety of other methods for non-invasive glucose monitoring are available that mostly include the use of light sensing techniques. These include optical rotation of polarized light wherein rotation of linearly polarized light contacts and travels through a person's skin; infrared light, including near-infrared spectroscopy (NIR), which may be utilized to monitor the blood glucose level of human subjects; and thermal infrared spectroscopy (TIR spectroscopy) utilized as a non-invasive method to monitor the blood glucose in a person. TIR is a subset of infrared spectroscopy that deals with radiation emitted in the infrared part of the electromagnetic spectrum. This method is commonly utilized to identify the composition of a surface, such a skin, by analyzing its spectrum and comparing it to previously measured materials.

Yet another non-invasive method includes impedance spectroscopy technology, which is a very versatile electrochemical tool to characterize intrinsic electronic properties of any material and its interface. The basis of impedance spectroscopy is the analysis of the impedance (resistance of alternating current) of the observed system in subject to the applied frequency and exciting signal. This analysis provides quantitative information about the conductance, the diabetic coefficient, the static properties of the interfaces of a system, and its dynamic change due to absorption or charge-transfer-phenomena.

Further, dielectric spectroscopy may be utilized as a non-invasive method for monitoring glucose. In particular dielectric spectroscopy measures the dielectric properties of a medium, such as skin, as a function of frequency.

Also, magneto-wave spectroscopy technology may be utilized to determine glucose levels inside blood vessels. Such a device is able to read blood glucose directly from a blood vessel, without puncturing the skin, through the use of a novel Photoacoustic (optical and sound-based) technology.

Beam-splitting optics (including linear polarized beams) may be utilized to measure a glucose level of a subject with a first beam reading a location on a skin of a subject under test and a second beam that combines with a reflection of the first beam as reflected from the skin. A portion of the reflection that is produced at or near an interface between the dermis and the subcutaneous of the skin optically interferes with the second beam to obtain a second optical measurement and a ratio is utilized to obtain a measurement of the glucose level in the dermis.

The above sensor technologies provide the ability to detect and analyze glucose levels without invading the skin surface. Principally, most technologies comprise an optical coupler for optically connecting a skin surface to the device that contains a plurality of zones, a light source for illuminating a skin surface with one or more wavelengths of electromagnetic radiation and a detector for detecting radiation emanating from said skin surface after illumination.

Other techniques, such as, reverse iontophoresis, utilize an electrical current applied to the skin. The current pulls out salt, which carries water, which in turn carries glucose. The glucose concentration of this extracted fluid is measured and is proportionate to that of blood.

One of the most promising approaches for non-invasive glucose monitoring is utilizing optical coherence tomography (“OCT”) technology. OCT is a optical signal acquisition and processing method allowing extremely high-quality, micrometer-resolution, three-dimensional images from within optical scattering media (e.g., biological tissue) to be obtained. In distinction with other optical methods, OCT, an interferometric technique, is able to penetrate significantly deeper into the scattering medium.

In other words, OCT is a biological tissue optical scanning technique that produces high resolution cross sectional images of optical reflectivity. OCT is based on the principle of utilizing a low-coherence interferometer, wherein information concerning various biological structures is extracted from the time delays of reflected signals. As such, OCT technology is able to provide images of biological tissue with micrometer resolution and determine glucose in blood, tissue and other biological samples. However, while OCT technology provides glucose monitoring in a relatively fast and non-invasive manner, it is primarily limited to medical applications, such as opthalmology, dermatology, cerebrum, dentistry and internal medicine.

Therefore, it is readily apparent that there is a need for a non-invasive and easy to utilize apparatus that that can incorporate a variety of sensing techniques to monitor a person's glucose concentration while driving.

### BRIEF SUMMARY OF THE INVENTION

Briefly described, the preferred embodiment overcomes the above-mentioned disadvantages and meets the recognized need for such an apparatus by providing a non-invasive glucose monitor comprising a processor, an indicating mechanism, and at least one sensor that is disposed proximate the skin of a driver when the driver's skin is in contact with a motor vehicle operational component. The non-invasive glucose monitor is utilized by the driver placing their skin on the sensor, wherein the sensor subsequently measures the driver's glucose concentration via optical coherence tomography which penetrates the skin to the bloodstream and takes a glucose reading of the blood, thereby subsequently analyzing the driver's glucose concentration via the processor and displaying the driver's glucose concentration via the indicator mechanism.

The apparatus of the preferred embodiment could potentially result in obviating the need for the afore-mentioned CMV Federal Exemption and for exemptions in other countries, so long as the apparatus is operative within the vehicle being driven, thus benefiting all drivers by the overall safety provided.

According to its major aspects and broadly stated, the preferred embodiment is an apparatus for monitoring glucose comprising a processor, an indicating mechanism and sensors that contact the skin of a person when the skin is in contact with a motor vehicle operational component, such as, for exemplary purposes only, the steering wheel of a vehicle. The sensor preferably comprises an optical coherence tomography sensor that non-invasively, selectively measures glucose concentration in the bloodstream through the skin surface of the person.

The sensor is in electrical communication with the processor, wherein the processor analyzes the glucose concentration in the person. The processor is selectively programmable with a range of glucose concentrations, and the processor compares the glucose concentration in the person to the range of glucose concentrations. If a condition is met, the processor communicates electrically with the indicating mechanism to provide information to the person operating the vehicle. The indicating mechanism comprises, for exemplary purposes only, meters, alarms, and combinations thereof. The meters display the glucose concentration in the person, and the alarms selectively signal an alert if the glucose concentration is outside of the range of pre-determined glucose concentrations.

In an alternate embodiment, the apparatus for monitoring glucose comprises non-invasive sensors disposed within an aftermarket component added to a motor vehicle, such as, for exemplary purposes only, a steering wheel cover, and again the sensors selectively comprise, without limitation, optical coherence tomography sensor, sensors based on glucokinase modulation that utilizes a catalytically disabled glucokinase protein, sensors based on optical rotation of polarized light, infrared light sensors, near-infrared spectroscopy sensors, thermal emission spectroscopy sensors, thermal infrared spectroscopy sensors, impedance spectroscopy sensors, dielectric spectroscopy sensors, mid-infrared ray technology sensors, magneto-wave spectroscopy sensors or photoacoustic sensors.

The preferred embodiment further comprises a method for monitoring glucose by obtaining an apparatus for monitoring glucose that comprises sensors, a processor and an indicator. The non-invasive sensors are disposed within an operational component of a motor vehicle, such as, for exemplary purposes only, a steering wheel, placing a portion of the driver's skin next to the sensors, measuring glucose concentration in the driver's bloodstream via optical coherence tomography, analyzing the driver's glucose concentration with the processor; and displaying the driver's glucose concentration via the indicator. The method may further comprise programming the processor with a range of glucose concentrations, and comparing the driver's glucose concentration with the range of glucose concentrations, subsequently signaling an alert to the indicator mechanism if the driver's glucose concentration is outside of the programmed range of glucose concentrations.

Additionally, the preferred embodiment is an apparatus for non-invasively determining a vehicle driver's glucose concentration having a sensor, such as, for exemplary purposes only, an optical coherence tomography sensor, disposed in the steering wheel of the vehicle and an alarm to let the driver know their glucose concentration, and the alarm is activated if the glucose concentration is outside of a selected range, such as, for exemplary purposes only 100 to 300 mg/dl, which according to the National Diabetics Information Clearinghouse (NDIC), blood glucose levels should be above 70 mg/dl and not stay above 180 mg/dl to prevent hypoglycemia. Hypoglycemia. (2008, October). Retrieved Feb. 3, 2009 from http://diabetes.niddk.nih.gov/dm/pubs/hypoglycemia. When Your Blood Glucose Is Too High or Too Low. (2006, October). Retrieved Feb. 3, 2009 from http://diabetes.niddk.nih.gov/dm/pubs/type1and2/lowglucose.htm.

More specifically, the preferred embodiment is a glucose monitor comprising a steering wheel, sensors disposed in the steering wheel or added to the steering wheel via an aftermarket device, a processor and an indicating mechanism that comprises a meter and/or an alarm. The sensors are located along the perimeter of the steering wheel. The processor is installed, for exemplary purposes only, within the dashboard of a motor vehicle, or alternately may be in a mobile container on the floor of the vehicle for use when an aftermarket sensor is utilized. It will be recognized by those skilled in the art that the processing equipment and the sensors may be disposed anywhere on the interior of the motor vehicle other than the dashboard or the steering wheel.

In use, a person places their hands along the steering wheel over the sensors. The optical coherence tomography sensors shine low coherence light into the microvasculature structure of the person utilizing a low-coherence interferometer, thereby producing high resolution cross-sectional images of the person's biological tissue to measure the person's glucose concentration from extracted time delays of reflected signals. The glucose concentration is subsequently sent to the processor via the sensor wiring, wherein the processor analyzes the glucose concentration and sends same to the indicating mechanism via the indicator wiring.

Additionally, the processor may be selectively programmed with a range of glucose concentrations acceptable for safe driving by the person, such as, for exemplary purposes only, between 100 and 300 mg/dl. The processor compares the glucose concentration in the person sent from the sensors to the programmed range of glucose concentrations. If the glucose concentration of the person is not within the range of programmed glucose concentrations, then the processor sends a signal to the alarm, alerting the person that he or she cannot operate a vehicle safely and/or may need to seek medical attention.

It will be recognized by those skilled in the art that the alarm may comprise any sort of indicator known in the art, such as, a digital alarm, an analog alarm, a sound alarm, a visual alarm, and/or the like. Further, it will be recognized by those skilled in the art that other non-invasive methods for monitoring glucose may be utilized other than OCT technology, such as, for exemplary purposes only, glucokinase modulation that utilizes a catalytically disabled glucokinase protein, optical rotation of polarized light, infrared light, near-infrared spectroscopy, thermal emission spectroscopy, thermal infrared spectroscopy, impedance spectroscopy, dielectric spectroscopy, mid-infrared ray technology, magneto-wave spectroscopy, photoacoustics, and the like. It will also be recognized by those skilled in the art that the glucose monitor may be utilized to monitor other physiological components of the person.

In an alternate embodiment, the glucose monitor is installed as an aftermarket addition to the motor vehicle, such as, for exemplary purposes only, a steering wheel cover having sensors installed therein. In use, the steering wheel cover is removably disposed over the steering wheel of the vehicle, and communicates electrically with the processor and indicating mechanism, which are also placed inside the vehicle in a selected location, such as, for exemplary purposes, on the top of the vehicle's dashboard, on the passenger's seat, or on the floor of the vehicle. It will be recognized by those skilled in the art that the steering wheel cover may be detachable and secured to the wheel by zippers, clasps, hook-and-loop fasteners, lacing, and the like.

Accordingly, a feature and advantage of the preferred embodiment is its ability to provide an apparatus and method for non-invasive blood glucose monitoring, thereby avoiding the inconvenience and risk associated with traditional invasive blood glucose monitoring techniques.

Another feature and advantage of the preferred embodiment is its ability to provide rapid results in sufficient time to administer appropriate medication.

Another feature and advantage of the preferred embodiment is its ability to continuously notify a driver of their current blood glucose concentration.

Yet another feature and advantage of the alternate embodiment is its ability to provide an apparatus and method that is portable and monitors a person's glucose concentration while driving.

Another feature and advantage of the preferred embodiment is its ability to provide a hands-free device for drivers to monitor their glucose concentration.

Yet still another feature and advantage of the preferred embodiment is its ability to alert a driver if their glucose level rises or falls beyond an acceptable level or range, thereby improving driver safety.

These and other features and advantages of the preferred embodiment will become more apparent to one skilled in the art from the following description and claims when read in light of the accompanying drawings.

### BRIEF DESCRIPTION OF THE SEVERAL VIEWS OF THE DRAWINGS

The preferred embodiment will be better understood by reading the Detailed Description of the Preferred and Selected Alternate Embodiments with reference to the accompanying drawing figures, in which like reference numerals denote similar structure and refer to like elements throughout, and in which:

FIG. 1 is a perspective view of a cutaway of the interior of a motor vehicle having a glucose monitor according to a preferred embodiment;

FIG. 2 is a perspective view of a cutaway of the interior of a motor vehicle with a glucose monitor according to a preferred embodiment, shown in use; and

FIG. 3 is a perspective view of a glucose monitor according to an alternate embodiment installed as an aftermarket device on the steering wheel of a motor vehicle.

### DETAILED DESCRIPTION OF THE PREFERRED AND SELECTED ALTERNATE EMBODIMENTS OF THE INVENTION

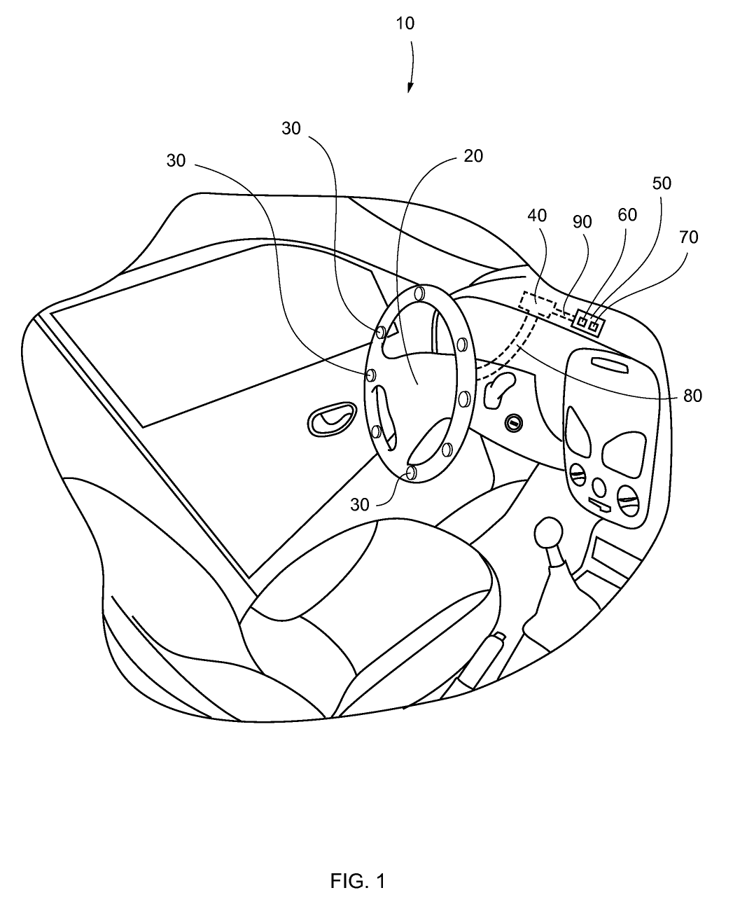

In describing the preferred and selected alternate embodiments, as illustrated in FIGS. 1-3, specific terminology is employed for the sake of clarity. The embodiments, however, are not intended to be limited to the specific terminology so selected, and it is to be understood that each specific element includes all technical equivalents that operate in a similar manner to accomplish similar functions.

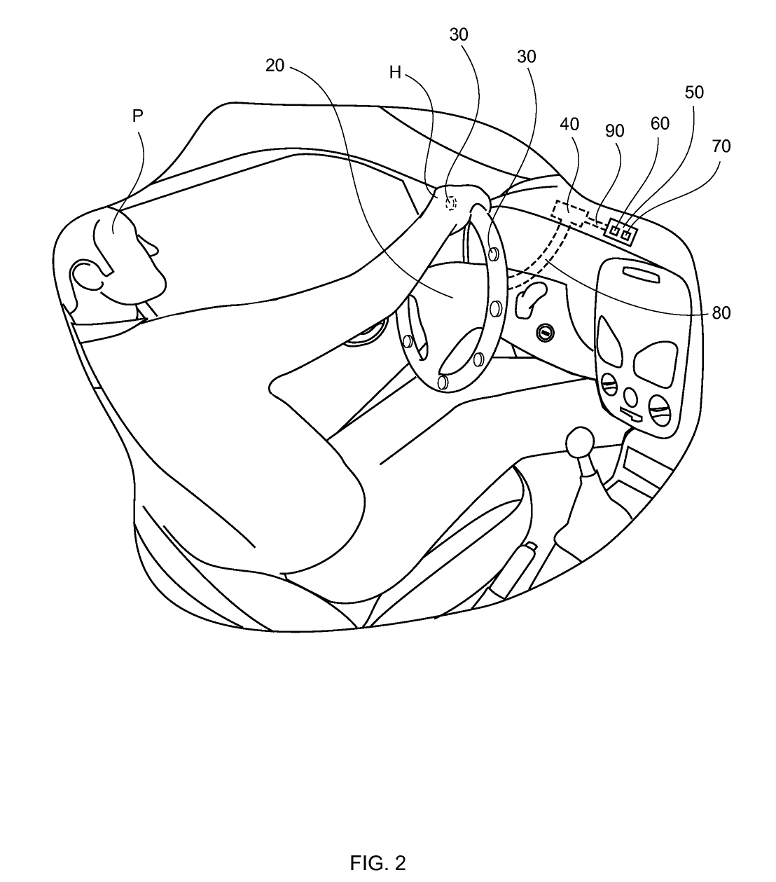

Referring now to FIG. 1, in a preferred embodiment glucose monitor 10 comprises steering wheel 20, sensors 30, processor 40 and indicating mechanism 50, and wherein indicating mechanism 50 preferably comprises meter 60 and/or alarm 70, and wherein alarm 70 preferably comprises an audible alarm, a visual alarm, or the like. It will be recognized by those skilled in the art that meter 60 and/or alarm 70 could be disposed in steering wheel 20, wherein meter 60 is more readily visible to driver P (best shown in FIG. 2) and wherein alarm 70 could provide tactile feedback to advise driver P of a selected condition as described more fully hereinbelow. Sensors 30 are preferably disposed along the perimeter of steering wheel 20 in a location where driver P will make contact with sensors 30 with his/her hands H while driving. Processor 40 is preferably disposed within the dashboard and is in electrical communication with sensors 30 via sensor wiring 80. Processor 40 is also in electrical communication with indicating mechanism 50 via indicator wiring 90. It will be recognized by those skilled in the art that processor 40 and sensors 30 may be disposed anywhere on the interior of the motor vehicle other than the dashboard or steering wheel 20 and it will be further recognized that glucose monitor 10 may be powered from the vehicle battery.

Referring now to FIG. 2, in use, person P places hands H on sensors 30, wherein sensors 30 comprise, for exemplary purposes only, optical coherence tomography sensor that radiate signal low coherence light into the microvasculature structure of hands H person P, and wherein sensors 30 measure the glucose concentration in person P. The glucose concentration in person P is subsequently sent to processor 40 via sensor wiring 80, wherein processor 40 analyzes the glucose concentration in person P. The glucose concentration of person P is then sent from processor 40 to indicating mechanism 50 comprising meter 60 via indicator wiring 90, wherein meter 60 displays the glucose concentration in person P.

Additionally, processor 40 is selectively programmable with a range of glucose concentrations acceptable for safe driving by person P, such as, for exemplary purposes only, between 100 and 300 mg/dl, wherein processor 40 compares the glucose concentration in person P sent from sensors 30 to the range of programmed glucose concentrations. If the glucose concentration of person P is not within the range of glucose concentrations, then processor 40 sends an alert to alarm 70 via indicator wiring 90, wherein alarm 70 alerts person P that he or she cannot operate a vehicle safely and/or may need to seek medical attention.

It will be recognized by those skilled in the art that alarm 70 may comprise any sort of indicator known in the art, such as, an audio alarm, a visual alarm, a digital alarm, an analog alarm, and the like, wherein the audio alarm produces a noise to alert the driver, the visual alarm displays a signal and/or message on indicating mechanism 50, the analog alarm provides an electronic pulse to indicating mechanism 50 and the digital alarm signals a numerical value to indicating mechanism 50. Further, it will be recognized by those skilled in the art that other non-invasive methods for monitoring glucose may be utilized other than OCT technology, such as, for exemplary purposes only, glucokinase modulation that utilizes a catalytically disabled glucokinase protein, optical rotation of polarized light, infrared light, near-infrared spectroscopy, thermal emission spectroscopy, thermal infrared spectroscopy, impedance spectroscopy, dielectric spectroscopy, mid-infrared ray technology, magneto-wave spectroscopy, photoacoustics, and the like. It will also be recognized by those skilled in the art that glucose monitor 10 may be utilized to monitor other physiological components of person P.

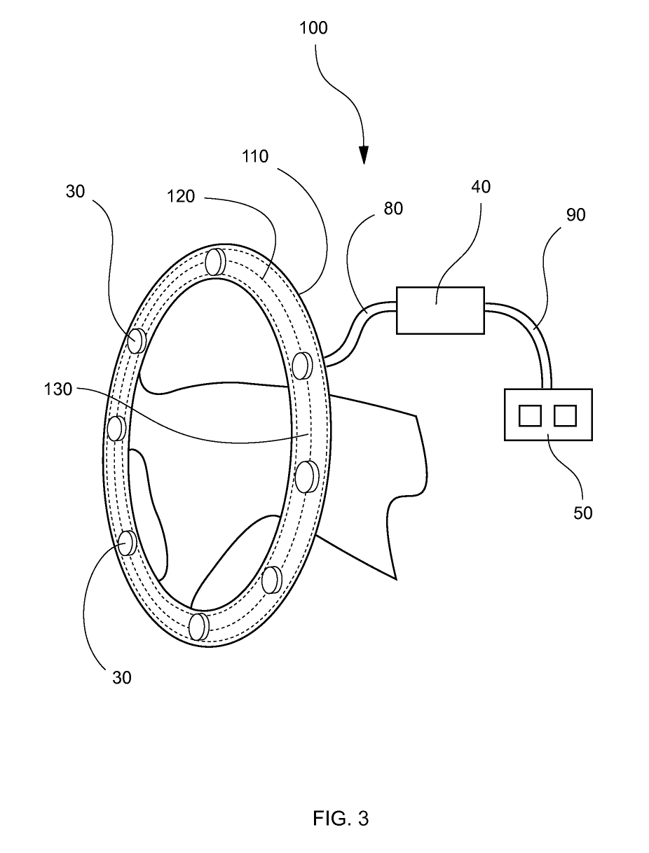

Referring now to FIG. 3, illustrated therein is an alternate embodiment of glucose monitor 10, wherein the alternate embodiment of FIG. 3 is substantially equivalent in form and function to that of the preferred embodiment detailed and illustrated in FIGS. 1-2 except as hereinafter specifically referenced. Specifically, the embodiment of FIG. 3 comprises glucose monitor 100, wherein glucose monitor 100 is installed as an after-market addition to a motor vehicle. Glucose monitor 100 comprises steering wheel cover 110, wherein steering wheel cover 110 comprises sensors 30 and fasteners 120, and wherein fasteners 120 allow steering wheel cover 110 to be easily fitted around, and secured to, steering wheel 130 of the vehicle.

In use, steering wheel cover 110 is disposed over steering wheel 130 of the vehicle, and processor 40, indicating mechanism 50, sensor wiring 80 and indicator wiring 90 are routed inside the vehicle, such as, for exemplary purposes, on the top of the vehicle's dashboard or on the passenger's seat. It will be recognized by those skilled in the art that steering wheel cover 110 is removable and may be secured to steering wheel 130 by any means known in the art, wherein fasteners 120 could comprise, for exemplary purposes only, zippers, clasps, hook-and-loop fasteners, lacing, and the like.

The foregoing description and drawings comprise illustrative embodiments of the preferred embodiment. Having thus described exemplary embodiments of the preferred embodiment, it should be noted by those skilled in the art that the within disclosures are exemplary only, and that various other alternatives, adaptations, and modifications may be made within the scope of the preferred embodiment. Merely listing or numbering the steps of a method in a certain order does not constitute any limitation on the order of the steps of that method. Many modifications and other embodiments will come to mind to one skilled in the art to which this preferred embodiment pertains having the benefit of the teachings presented in the foregoing descriptions and the associated drawings. Although specific terms may be employed herein, they are used in a generic and descriptive sense only and not for purposes of limitation. Accordingly, the preferred embodiment is not limited to the specific embodiments illustrated herein, but is limited only by the following claims.

What is claimed is:

1. An apparatus for monitoring glucose comprising:
at least one non-invasive sensor, wherein said at least one sensor is disposed proximate the skin of a person when said skin is in contact with a motor vehicle operational component;
a processor; and
an indicating mechanism.

2. The apparatus of claim 1, wherein said motor vehicle operational component comprises a steering wheel.

3. The apparatus of claim 1, wherein said at least one sensor comprises an optical coherence tomography sensor.

4. The apparatus of claim 3, wherein said optical coherence tomography sensor selectively measures glucose concentration through a skin surface of the person.

5. The apparatus of claim 4, wherein said at least one sensor is in electrical communication with said processor.

6. The apparatus of claim 5, wherein said processor analyzes said glucose concentration in the person.

7. The apparatus of claim 6, wherein said processor is selectively programmable with a range of glucose concentrations.

8. The apparatus of claim 7, wherein said processor compares said glucose concentration in the person to said range of glucose concentrations.

9. The apparatus of claim 8, wherein said processor is in electrical communication with said indicating mechanism.

10. The apparatus of claim 9, wherein said indicating mechanism comprises an indicator selected from the group consisting of meters, alarms, and combinations thereof.

11. The apparatus of claim 10, wherein said meters selectively display said glucose concentration.

12. The apparatus of claim 10, wherein said alarms selectively signal an alert if said glucose concentration is outside of said range of glucose concentrations.

13. The apparatus of claim 1, wherein said at least one sensor is disposed within an aftermarket component added to a motor vehicle.

14. The apparatus of claim 13, wherein said aftermarket component comprises a steering wheel cover.

15. The apparatus of claim 1, wherein said at least one sensor comprises a sensor selected from the group consisting of optical coherence tomography, glucokinase modulation that utilizes a catalytically disabled glucokinase protein, optical rotation of polarized light, infrared light, near-infrared spectroscopy, thermal emission spectroscopy, thermal infrared spectroscopy, impedance spectroscopy, dielectric spectroscopy, mid-infrared ray technology, magneto-wave spectroscopy, photoacoustic, and the like.

16. A method for monitoring glucose, said method comprising the steps of:
obtaining an apparatus for monitoring glucose, wherein said apparatus comprises at least one sensor, a processor and an indicator mechanism, and wherein said at least one sensor is disposed within an operational component of a motor vehicle;
disposing a portion of a person's skin proximate said at least one sensor;
non-invasively measuring the person's glucose concentration via optical coherence tomography;
analyzing the person's glucose concentration via said processor; and
displaying the person's glucose concentration via said indicator mechanism.

17. The method of claim 16, said method further comprising the steps of:
programming said processor with a range of glucose concentrations;
comparing the person's glucose concentration with said range of glucose concentrations; and
signaling an alert to said indicating mechanism if the person's glucose concentration is without said range of glucose concentrations.

18. An apparatus for determining a vehicle driver's glucose concentration, said apparatus comprising:
a non-invasive sensor disposed in a steering wheel of the vehicle, wherein said sensor monitors the driver's glucose concentration; and
an alarm, wherein said alarm is activated if said glucose concentration is outside of a selected range.

19. The apparatus of claim 18, wherein said sensor comprises optical coherence tomography.

20. The apparatus of claim 18, wherein said range comprises 100 to 300 mg/dl.
